(19) United States
(12) Patent Application Publication
(10) Pub. No.: US 2014/0113365 A1
(43) Pub. Date: Apr. 24, 2014

(54) CELL CULTURING VESSEL

(71) Applicants: Yusuke Nagai, Okayama-shi (JP); Keiji Naruse, Okayama-shi (JP); Ken Takahashi, Okayama-shi (JP); Eijiro Tokuyama, Okayama-shi (JP)

(72) Inventors: Yusuke Nagai, Okayama-shi (JP); Keiji Naruse, Okayama-shi (JP); Ken Takahashi, Okayama-shi (JP); Eijiro Tokuyama, Okayama-shi (JP)

(73) Assignees: NATIONAL UNIVERSITY CORPORATION OKAYAMA UNIVERSITY, Okayama-shi, Okayama (JP); MENICON CO., LTD., Nagoya-shi, Aichi (JP)

(21) Appl. No.: 14/114,134

(22) PCT Filed: Apr. 26, 2012
(86) PCT No.: PCT/JP2012/061271

§ 371 Date: Jan. 2, 2014

(30) Foreign Application Priority Data
Apr. 27, 2011 (JP) 2011-099776

(51) Int. Cl. C12M 1/00

(52) U.S. Cl. CPC C12M 23/26
USPC 435/305.1

(57) ABSTRACT

A cell culture vessel supplies cells close to the center of gel and deeper down to the bottom with sufficient nutrients for attaining an enhanced survival rate of the cultivated cells. The cell culturing vessel comprises a culture tray capable of holding a liquid culture medium and a cell mixture container fixed to inner walls of the culture tray for retaining the cell mixture. Once the culture tray is filled with the liquid culture medium, the cell mixture retained in the cell mixture container has its opposite outer parts exposed to the liquid culture medium.

### FIELD OF THE INVENTION

The present invention relates to cell culturing vessels that are capable of cultivating cells in a gel at an increased survival rate.

### BACKGROUND ART

Typical two-dimensional cell culture in the prior art employs vessels like a plastic culture plate.

As to materials suitable for scaffolds in three-dimensional cell culture, gels such as collagen have often been used to mix with cells into prepared cell-suspended gel formulas that are to be soaked into liquid culture media. In one method, a cell-suspended gel put in a plastic culture plate may be soaked with liquid culture medium; or otherwise, the cell-suspended gel, which is instead put in a three-dimensional incubator (see Patent Document 1 identified below) and spread therein to apply mechanical stimulus to suspended cells, may be soaked with the liquid culture medium, and if necessary, the three-dimensional incubator stretchable in itself is extended during cultivating the cells.

The three-dimensional incubator, which cradles the gel serving as scaffolds for the cells, is then required to apply stress on the cells uniformly. To that end, the incubator is shaped in a rectangular box of a deformable material and has a bottom membrane and side walls contiguous to and upright from the entire peripheral edge of the bottom membrane, and the inner surfaces of the side walls are porous.

### DOCUMENT OF THE RELATED ART

### Patent Document

Patent Document 1: Repub. of PCT Intl. Pub. No. WO 2007/123035

### SUMMARY OF THE INVENTION

### Problem to be Solved by the Invention

With any of the aforementioned cell culturing vessels in the prior art, namely the plastic culture plates and the existing three-dimensional incubator (Patent Document 1), only an upper surface of the cell-suspended gel comes in contact with the liquid culture media that supply the cells with nutrients, that is, only the cells in part of the gel exposed to the liquid culture media are benefitted from the nutrients, resulting in the remaining close to the center of the gel and even deeper down to the bottom being nurtured insufficiently, which brings about a reduced survival rate of the cultivated cells.

### Object of the Invention

The present invention is made to overcome the above-mentioned disadvantage in the prior art cell culturing vessels, and accordingly, it is an object of the present invention to provide a cell culture vessel capable of supplying cells close to the center of gel and deeper down to the bottom with sufficient nutrients for attaining an enhanced survival rate of the cultivated cells.

### Solution to the Problem

The present invention provides a cell culturing vessel comprising a culture tray capable of holding a liquid culture medium and a cell mixture container fixed to inner walls of the culture tray for retaining the cell mixture.

Once the culture tray is filled with the liquid culture medium, the cell mixture retained in the cell mixture container has its opposite outer parts exposed to the liquid culture medium.

### Effect of the Invention

The cell culturing vessel of the present invention attains an enhanced survival rate of cultivated cells and facilitates spreading cell-suspended gel so as to more easily and effectively apply mechanical stimulus to the cells.

### Various Aspects of the Invention

In the aforementioned solution to the prior art disadvantage, the culture tray and the cell mixture container are deformable.

In the aforementioned solution, the opposite outer parts of the cell mixture include top and bottom surfaces of the cell mixture.

In the aforementioned solution, the opposite outer parts of the cell mixture include lateral or longitudinal sides of the cell mixture.

The opposite outer parts of the cell mixture include top and bottom surfaces and lateral or longitudinal sides of the cell mixture.

In the aforementioned solution, the cell mixture is a cell-suspended gel.

Arranged in this manner, the cell culture vessel permits the cell-suspended gel to be externally spread so as to more easily and effectively apply mechanical stimulus to the cultivated cells.

### BRIEF DESCRIPTION OF THE DRAWINGS

FIG. 1 is a perspective view showing a first embodiment of a cell culturing vessel according to the present invention.

FIG. 2 is a sectional view taken along the line II-II of FIG. 1.

FIG. 3 is a perspective view showing a second embodiment of the cell culturing vessel according to the present invention.

FIG. 4 is a sectional view taken along the line IV-IV of FIG. 3.

FIG. 5 is a top plan view showing a third embodiment of the cell culturing vessel according to the present invention.

FIG. 6 is a perspective view showing a fourth embodiment of the cell culturing vessel according to the present invention.

FIG. 7 is a sectional view taken along the line VII-VII of FIG. 6.

FIG. 8 is a sectional view taken along the line VIII-VIII of FIG. 6.

FIG. 9 is a sectional view showing a prior art typical cell culturing vessel used for a control experiment.

FIG. 10 is a photograph of the control experiment, showing a gel cradled in the cell culturing vessel provided as the first embodiment of the present invention.

FIG. 11 is another photograph of the control experiment, showing a gel cradled in the prior art typical cell culturing vessel.

### BEST MODE OF THE INVENTION

### Embodiment 1

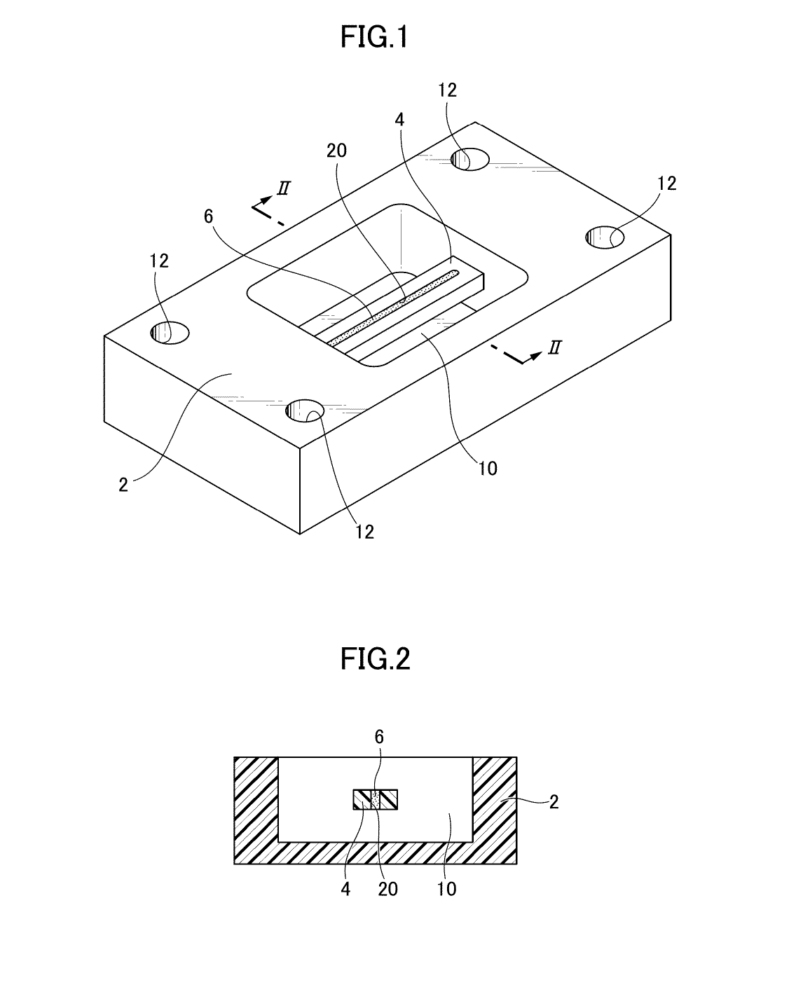

A first embodiment of a cell culturing vessel according to the present invention comprises, as shown in FIGS. 1 and 2, a culture tray 2, a gel container 4 holding gel or cell mixture, and gel 6 in which cells are suspended.

The culture tray 2 may be made of any of materials such as silicon elastomer, PDMS (polydimethylsiloxane), and the like. The culture tray 2 may be a commercially available culture chamber STB-CH-04 from Strex Incorporation, JPN. The Strex culture chamber STB-CH-04 is shaped almost like a rectangular parallelepiped of 25 mm in lengthwise dimension, 40 mm in lateral dimension, and 12 mm in height, and has a center recess defined as a liquid culture medium well 10 of 20 mm in lengthwise dimension, 20 mm in lateral dimension, and 10 mm in depth. In four corners of the culture tray 2, provided are holes 12 for screws by which the culture tray 2 is fixed down on a stress applying device (not shown). The stress apply device (not shown) is suitable for applying stress to the culture tray 2 to deform the culture tray 2 and the gel container 4 detailed below.

The gel container 4 may be formed by cutting silicon foam sheet (SSP-2.0S and SSP-4.0S) available from AS ONE Corporation, JPN. The gel container 4 is dimensioned to be approx. 1.5 mm in lengthwise dimension, approx. 1.2 mm in height, and 20 mm in lateral dimension that is equal to the lateral dimension of the liquid culture medium well 10. In the center along the lateral extension of the gel container 4, defined is a gel well 20 that is a cut as narrow as approx. 1.0 mm and left open at both of its top and bottom ends without being blocked by the bottom of the well 10.

The gel container 4 is bonded to the laterally opposite inner walls of the culture tray 2 by which the liquid culture medium well 10 is defined. For that purpose, suitable adhesives include silicon resin products TSE3032(A) and TSE3032(B) commercially available from Momentive Performance Material Japan Inc., and in use, a solution of TSE3032(A) and TSE3032(B) mixed with a rate of 10 to 1 is applied to joints of both the components and heated at 60 degrees centigrade for an hour.

The gel 6 in which cells are suspended in three-dimensional dispersion may be self-organizing peptide gel, collagen gel, or the like. The gel 6 is loaded within the gel well 20 in the gel container 4.

### Embodiment 2

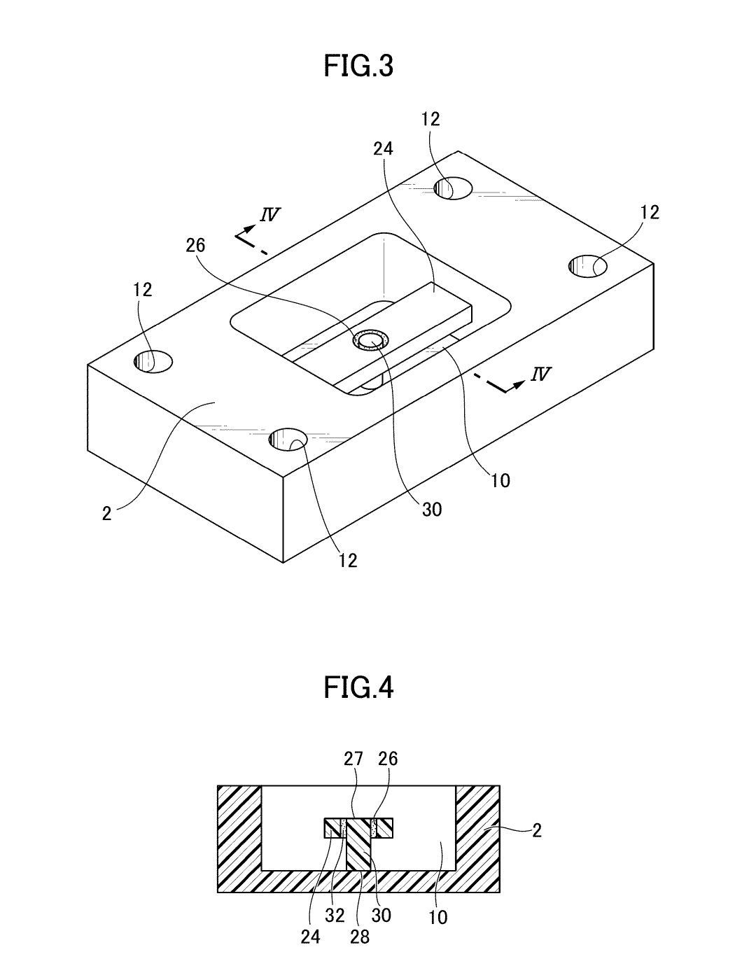

A second embodiment of the cell culture vessel will now be described with reference to FIGS. 3 and 4 where components similar in structure to those of the first embodiment are denoted by like reference numerals, and the description of such components is omitted.

A gel container 24 has a circular gel well 26 that is a center cut circular in shape and left open at both of its top and bottom ends. The circular gel well 26 surrounds a support pole member 30 having its upper end 27 flush with the upper surface of the gel container 24 and its lower end 28 flush with the bottom surface of the liquid culture medium well 10. The gel container 24 is spaced from the support pole member 30 by a certain distance, being defined as an opening 32 which is filled with the gel 6.

### Embodiment 3

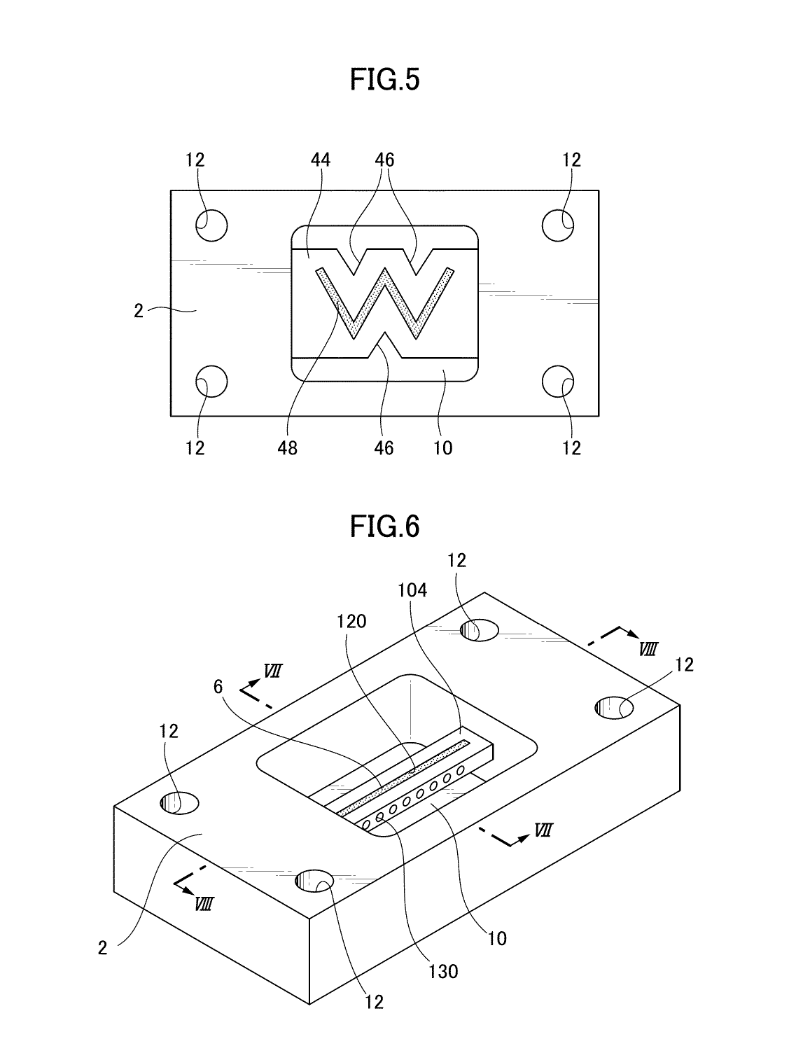

A third embodiment of the cell culture vessel will now be described with reference to FIG. 5 where components similar in structure to those of the first embodiment are denoted by like reference numerals, and the description of such components is omitted.

Similar to the gel container 14 in the first embodiment of the present invention, a gel container 44 is fixed so as to be apart from the bottom of the liquid culture medium well 10. The gel container 44 is not so narrow as the gel container 14 in the first embodiment and is provided with several V-shaped notches 46 along the opposite edges. In the center of the gel container 44, an open-ended W-shaped gel well 48 is defined.

### Embodiment 4

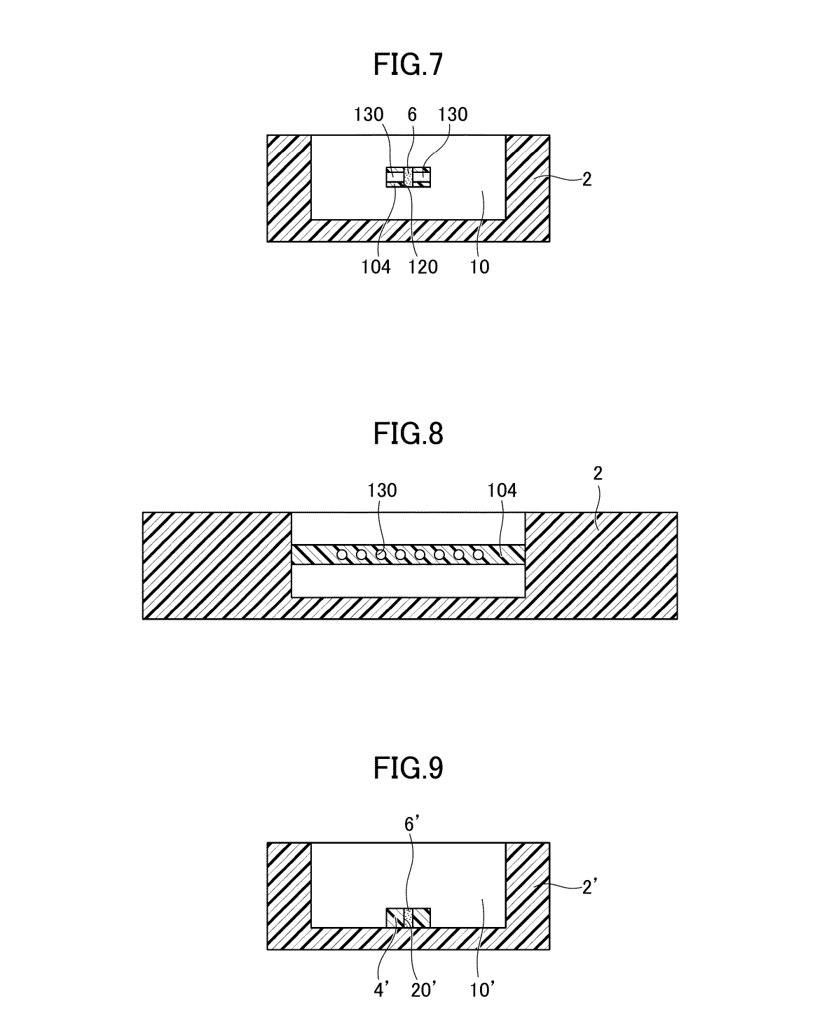

A fourth embodiment of the cell culture vessel will now be described with reference to FIGS. 6 to 8 where components similar in structure to those of the first embodiment are denoted by like reference numerals, and the description of such components is omitted.

The gel container 104 is formed of silicon resin products TSE3032(A) and TSE3032(B) commercially available from Momentive Performance Material Japan Inc. A solution of TSE3032(A) and TSE3032(B) mixed with a rate of 10 to 1 is injected in a mold and then heated at 60 degrees centigrade for two hours to have a cured piece of silicon resin cast. The gel container 104 is 3.0 mm in lengthwise dimension, 2.0 mm in height, and 20 mm in lateral dimension identical to a lateral dimension of the liquid culture medium well 10. In the center along the lateral extension of the gel container 104, defined is a gel well 120 that is a cut as narrow as approx. 1.0 mm and left open at both of its top and bottom ends. The gel container 104 further has horizontal open-ended orifices 130 of 1.0-mm diameter that extend horizontally and cross the gel well 120. The horizontal open-ended orifices 130 permit anterior/posterior sides of the gel 6 loaded in the gel well 120 and thus cells around the opposite sides to be exposed partially to the liquid culture medium.

The gel container 104 is bonded to the laterally opposite inner walls of the culture tray 2 by which the liquid culture medium well 10 is defined. For that purpose, suitable adhesives include the silicon resin products TSE3032(A) and TSE3032(B) commercially available from Momentive Performance Material Japan Inc., and in use, a solution of TSE3032(A) and TSE3032(B) mixed with a rate of 10 to 1 is applied to joints of both the components and heated at 60 degrees centigrade for an hour.

Control Experiment

In order to observe that the cell culturing vessel of the present invention is able to attain an increased survival rate of cultivated cells by virtue of sufficient nutrient supply to the cells suspended around the center of the gel and even deeper down to the bottom of the gel, a control experiment as detailed below was carried out.

A first sample of the cell culturing vessel is the one that has been described as the first embodiment of the present invention. A second sample of the cell culturing vessel, as shown in FIG. 9, has its gel container 4′ along with gel 6′ therein brought in contact with the bottom surface of a liquid culture medium well 10′, and a supposition took place that cells suspended in the gel 6′ would not be supplied with sufficient nutrients from the liquid culture medium.

In advance of cultivation in the first and second samples of the cell culturing vessel, mouse myoblast cells (C2C12) are mixed with self-assembling peptide gel (PanaceaGel SPG178 from Menicon Life Science, JPN) to have a cell-suspended gel preparation of 2×10⁶ cells/mL (the final concentration of SPG178 was 0.27%). A pipette was used to transfer 60 μL of the mix or the cell-suspended gel into the gel well cut open in the silicon foam sheet in each of the cell culturing vessels. Succeedingly, 3 mL of a liquid culture medium (DMEM+10% FCS) was poured to get the mix or the cell-suspended gel immersed, and then, both the cell culturing vessels were left in an incubator keeping the temperature at 37 degrees centigrade (filled with the ambient air containing 5% of CO₂) for three-day cultivation.

Three days after, the liquid culture medium was eliminated from the well, and instead applied is approx. 3 mL of DMEM in which a live cell staining pigment (Calcein-AM from Dojindo Laboratories, JPN) is solved to have 8.3-μg/mL pigment preparation, and both of the cell culturing vessels were left in the incubator keeping the temperature at 37 degrees centigrade (with the ambient air containing 5% of CO₂) for 30 minutes.

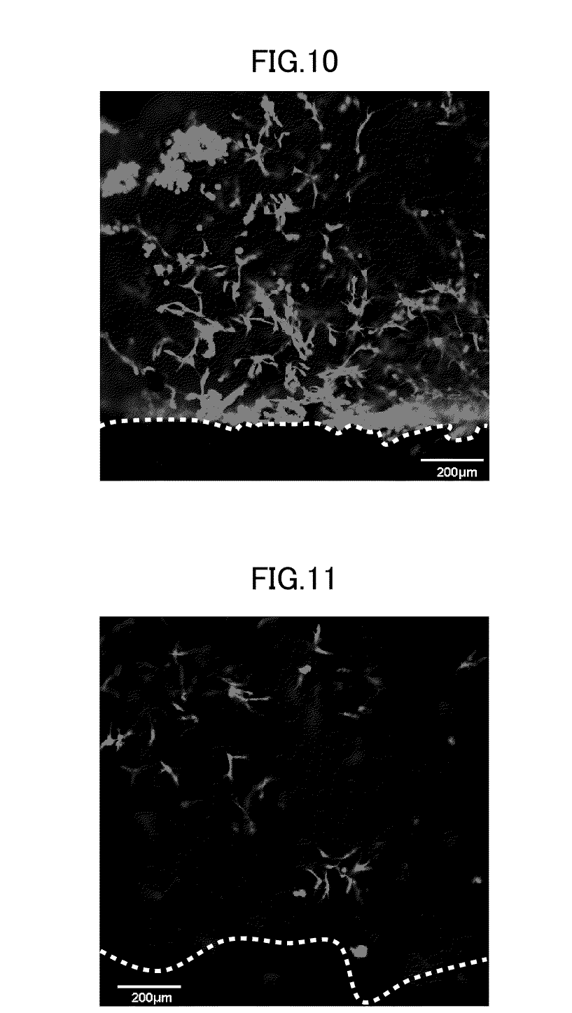

30 minutes after, the gel was removed from the gel well cut open in the silicon foam sheet, and then, it was cross-sectioned by using a scalpel for the succeeding observation of a live cell distribution in a gel lamina around the bottom under a confocal laser microscope FLUOVIEW FV1000 available from Olympus Corp., JPN. FIG. 10 is a laminagram of the gel derived from the first sample of the cell culture vessel identical with the first embodiment according to the present invention. FIG. 11 is another laminagram of the gel derived from the second sample of the cell culture vessel typical in the prior art. In these stained object targeting images, white dots are live cells, and a broken line shows the lower boarder or the bottom line of the gel. A conclusion drawn from the stained object targeting images is that the first sample of the cell culture vessel identical with the first embodiment according to the present invention attains a considerably enhanced cell survival rate than the second sample of the cell culture vessel typical in the prior art.

### DESCRIPTION OF THE REFERENCE NUMERALS

2 Cell Culture Vessel
4 Gel Container
6 Gel
10 Liquid Culture Medium
12 Screw Holes
20 Gel Well
24 Gel Container
26 Circular Gel Well
27 Upper End of Support Pole Member
28 Lower End of Support Pole Member
30 Support Pole Member
32 Opening
44 Gel Container
48 W-Shaped Gel Well
104 Gel Container
120 Gel Well
130 Horizontal Open-Ended Orifices

1. A cell culturing vessel comprising a culture tray capable of holding a liquid culture medium and a cell mixture container fixed to inner walls of the culture tray for retaining the cell mixture,
once the culture tray is filled with the liquid culture medium, the cell mixture retained in the cell mixture container having its opposite outer parts exposed to the liquid culture medium,
the culture tray and the cell mixture container being deformable.

2. (canceled)

3. The cell culturing vessel according to claim 1, wherein the opposite outer parts of the cell mixture include top and bottom surfaces of the cell mixture.

4. The cell culturing vessel according to claim 1, wherein the opposite outer parts of the cell mixture include lateral or longitudinal sides of the cell mixture.

5. The cell culturing vessel according to claim 1, wherein the opposite outer parts of the cell mixture include top and bottom surfaces and lateral or longitudinal sides of the cell mixture.

6. The cell culturing vessel according to claim 1, wherein the cell mixture is a cell-suspended gel.
